(19) United States
(12) Patent Application Publication
(10) Pub. No.: US 2005/0191353 A1
(43) Pub. Date: Sep. 1, 2005

(54) Process for manufacture of stable oral multiple unit pharmaceutical composition containing benzimidazoles

(76) Inventors: Amit Krishna Antarkar, Nagpur (IN); Abdul Sattar Abdul Jawed, Buldana (IN); Parag Narayan Gadkari, Thane (West) (IN); Gaurang Hasmukhlal Thanawala, Thane (IN); Maya Janak Shah, Mumbai (IN); Janak Ramanlal Shah, Mumbai (IN); Rajendra Ghanshamlal Lala, Mumbai (IN); Ketaki Kishore Joshi, Mumbai (IN)

Correspondence Address: WINSTON & STRAWN LLP, 1700 K STREET, N.W., WASHINGTON, DC, 20006, US

(21) Appl. No.: 11/057,386

(22) Filed: Feb. 15, 2005

Related U.S. Application Data
(63) Continuation of application No. PCT/IB03/03514, filed on Aug. 4, 2003.

(30) Foreign Application Priority Data
Aug. 16, 2002 (IN) 742/MUM/2002

(51) Int. Cl. A61K 9/26; A61K 9/36

(52) U.S. Cl. 424/470; 427/2.14

(57) ABSTRACT

This invention relates to a process for the manufacture of a stable, oral, multiple unit pharmaceutical composition containing high concentrations of benzimidazole up to about 35 to 45% w/w, preferably up to about 40% w/w, a disintegrating agent, and one or more fillers. Surfactants in these compositions are in an enteric polymer layer and preferably not in contact with the benzimidazole. The process preferably involves sequential deposition of: (a) an alkaline material layer on non-pariel seeds to obtain treated non-pariel seeds; (b) a drug layer of the benzimidazole to obtain drug pellets; (c) a sealant polymer layer to obtain sealed pellets; and (d) an enteric polymer layer to obtain enteric-coated pellets.

### CROSS-REFERENCE TO RELATED APPLICATION

This application is a continuation of International Application PCT/IB2003/003514, filed Aug. 4, 2003, the entire content of which is expressly incorporated herein by reference.

### BACKGROUND OF THE INVENTION

Benzimidazolic compounds such as omeprazole, lansoprazole, pantoprazole, pariprazole, leminoprazole, and rabeprazole are potent proton pump inhibitors known for inhibition of gastric acid secretion. They are used in the therapy of diseases related to gastric acidity in mammals, especially in humans, including gastric and duodenal ulcers, reflux oesophagitis, gastritis, duodenitis and Zollinger-Ellison syndrome. Benzimidazoles such as omeprazole and lansoprazole are sensitive to light, heat and moisture. They exhibit fast decomposition below a pH of 7.8 and have a maximum stability at a pH of 11. Hence, the oral dosage containing benzimidazole needs to be protected from the acidic ingredient used to manufacture the dosage and from acidic gastric fluid so that it reaches the small intestine intact from where it is absorbed systemically. Benzimidazole also has very low aqueous solubility and its solubility is pH dependent. Various related art have addressed and have tried to overcome the following issues:
Stabilizing benzimidazole and protecting benzimidazole from acidic ingredients and acidic and physiological environments.
Releasing benzimidazole from enteric-coated pellets, preferably more than 85% w/w, in a pH of 6.8 after 45 minutes with minimum acid degradation in 0.1N HCl after 2 hours.
Manufacturing pharmaceutical compositions containing benzimidazole in single equipment without involving the use of organic solvents for making the process operator- and environment-friendly.

U.S. Pat. No. 4,786,505 and EP 0502556 A1 describe an extrusion spheronization process for benzimidazole, such as omeprazole preparation, wherein an omeprazole core is prepared by granulating a mixture of mannitol powder with lactose anhydrous, hydroxypropylcellulose and microcrystalline cellulose with water containing an alkaline compound and a surfactant.

The essential teachings of these patents are:
Use of a surfactant, disintegrating agent and filler in the composition, in contact with omeprazole, to achieve a desired dissolution of omeprazole.
Efficient drying until the water content of the final dosage form is less than 1.5% w/w, which is an absolute requirement for the stable formulation.

The optional feature of these patents is:
Use of pH buffering compounds in the separating layer that separates the omeprazole core and the enteric layer.

EP 1213015 A1 discloses a delayed controlled release pantoprazole composition. The process requires coating of propylene glycol, water-soluble alkaline material, sodium hydroxide and hydroxypropylmethylcellulose (HPMC) on sucrose pellets to obtain starter pellets. Example 3 disclosed in the specification indicates high concentration of HPMC (binder) about 100% w/w and propylene glycol about 50% w/w of pantoprazole sodium for its deposition along with an alkaline substance to obtain active pellets. The active pellets are further coated with at least one intermediate layer comprising a water-insoluble release-slowing film former and a member selected from the group consisting of a pore former, a plasticizer, a buffer, and a base and pigment dissolved or dispersed in a non-aqueous organic solvent. The aqueous insolubility of the intermediate layer is an absolute requirement and is critical for the application of the enteric layer in the form of an aqueous suspension. The process requires an essential use of fillers and disintegrants in the preparation of the tablet cores that are in contact with pantoprazole to enhance its dissolution.

U.S. Pat. No. 5,385,739 describes a microgranule dosage form containing omeprazole. A powder mixture of omeprazole, mannitol, a disintegrating agent (carboxymethylstarch) and a surfactant (sodium lauryl sulfate) is layered on a neutral core of sugar and starch in a circular turbine with an inclined flat bottom. The core further contains a protective layer consisting of mannitol, sucrose and a binding agent, which isolate the active agent from the external enteric coating layer. Omeprazole and the excipients that are used require an additional micronization step to achieve a particle size below 100 microns. Efficient drying is essential to reduce the moisture level of the active layer to preferably less than 0.5% to ensure good stability. The process also requires stringent temperature control to maintain the granules between 32° C. and 38° C.

U.S. Pat. No. 6,077,541 describes a process for the manufacture of omeprazole enteric-coated pellets wherein the drug layer comprises of 20-70% w/w omeprazole. The enteric coating layer is directly applied to the drug layer. The process requires the additional step of micronization of omeprazole. The process also requires alkaline agents, surface-active agents and binders, which along with micronized omeprazole, is suspended in aqueous or non-aqueous solvents. This suspension that contains omeprazole is coated on a non-pariel sugar sphere in a fluid bed coater.

EP 1108425 describes a multi-unit pharmaceutical preparation manufactured by coating an aqueous suspension of substituted benzimidazole, or its acceptable salts, in the micronized form onto an inert core to obtain a drug core. The invention requires the use of surfactants and disaggregants in the layer-containing drug, which are mixed in a suitable proportion to allow disaggregation of the formulations and dissolution of the active ingredients. The drug core, in turn, is coated with an insulating layer, free from alkaline agents, and of minimum thickness of 15-μm. Finally, a gastro-resistant layer is applied having a minimum thickness of 30-μm. The formulation has a stringent requirement that the humidity is less than 1.5% for good stability.

PCT Publication WO98/19668 describes a delayed drug delivery system where micronized omeprazole in an aqueous suspension containing a water-soluble binder is deposited on a core containing an alkaline material. The alkaline core structure is obtained by depositing a powder blend of an alkaline material and a spheronizing/disintegrating agent on non-pariel seeds with the help of a polymeric binding agent in a fluid bed granulator with a rotor insert. The drug core is further coated with a non-enteric moisture barrier followed by a delayed-release enteric barrier. The formulation releases only 60-80% of omeprazole after 45 minutes in a buffer having a pH of 6.8.

U.S. Pat. No. 6,207,198 describes an omeprazole composition where an alkaline reacting substance is not present in the composition. It describes a process that involves granulating omeprazole with inert nuclei or aqueous layering of suspended omeprazole onto inert nuclei. The granules or the layered nuclei are compressed to form microtablets, which are coated with an intermediate layer followed by an enteric layer. The composition makes use of disintegrants and surfactants in the intermediate layer, which is in immediate contact with a core containing omeprazole to improve the dissolution of omeprazole. The process is operator dependant and makes use of multiple equipment making it laborious and time consuming.

U.S. Application Publication No. 2001/0053387 describes an omeprazole composition where omeprazole, along with an alkaline material, is layered on non-pariel seeds in a centrifugal coater. These drug cores are then coated with a moisture barrier that contains a hydrophobic material (polydimethylsiloxane), followed by an enteric coating. The pellets are compressed into tablets or filled into hard gelatin capsules. Thus, the related prior art teaches:
To obtain the desired dissolution of benzimidazole from pharmaceutical compositions containing less than 15% w/w of benzimidazole, the benzimidazole needs to be distributed over a wide surface area.
To obtain the desired dissolution of benzimidazole from pharmaceutical compositions containing a high concentration of benzimidazole, the benzimidazole specifically utilized is in contact with a surfactant, a disintegrating agent, a filler or their mixtures.
Micronization of benzimidazole is absolutely essential.
Stringent requirement to have moisture content less than 1.5% w/w, which is essential for a stable formulation thereby increasing the drying time and processing time.
Essential use of a water-insoluble intermediate layer in an organic solvent to overcome the criticality during the application of the enteric layer in the form of an aqueous suspension.
Essential use of a high concentration of a binder such as HPMC about 100% w/w and propylene glycol about 50% w/w of benzimidazole in contact with benzimidazole.
Essential use of propylene glycol for the preparation of starter pellets and presealed pellets.
Essential use of a suspension of micronized benzimidazole for deposition leading to low batch yields, thereby making the product expensive.

None of the related art teaches a process for manufacturing of a stable, oral, multiple-unit pharmaceutical composition containing a high concentration of benzimidazole up to about 40% w/w without the use of micronized benzimidazole, disintegrating agents, fillers, and surfactants in contact with benzimidazole, and having a minimum acid degradation in 0.1N HCl after two hours and a buffer release of more than 85% w/w in a pH of 6.8 after 45 minutes in a single equipment fluid bed bottom spray processor and capable of being filled in smaller size capsules for ease of administration and patient acceptance.

Thus, there is a need for improvements to the prior art processes and resulting pharmaceutical compositions, and such improvements are now provided by the present invention.

### SUMMARY OF THE INVENTION

In one embodiment, the present invention relates to a process for manufacture of a pharmaceutical composition. This process comprises depositing, on non-pariel seeds, an alkaline material layer comprising a water-insoluble alkaline material to obtain treated non-pariel seeds with increased resistance to breakage; depositing, on the treated non-pariel seeds, a drug layer comprising benzimidazole in an amount of up to about 40% w/w of the composition and being substantially free of propylene glycol to obtain drug pellets; depositing, on the drug pellets, a sealant polymer layer which is substantially free of propylene glycol to obtain sealed pellets; and depositing, on the sealed pellets, an enteric polymer layer containing surfactants to obtain a pharmaceutical composition comprising enteric-coated pellets that are substantially free of surfactants, disintegrating agents, or fillers in contact with the benzimidazole.

The invention also relates to a pharmaceutical composition that is obtainable from or advantageously, is obtained from, the process described herein. This stable, oral, multiple-unit pharmaceutical composition is preferably in the form of enteric-coated pellets comprising non-pariel seeds coated with an alkaline material layer comprising a water-insoluble alkaline material; a drug layer, disposed over the alkaline material layer, comprising benzimidazole in an amount of up to about 40% w/w of the composition and being substantially free of propylene glycol; a sealant polymer layer, disposed over the drug layer, which is substantially free of propylene glycol; and an enteric polymer layer, disposed over the sealant polymer layer, containing surfactants, sequentially deposited on non-pariel seeds. In a preferred embodiment, at least a portion of each layer contacts the previous layer or is on the previous layer. The pharmaceutical composition is substantially free of surfactants, disintegrating agents, or fillers in contact with the benzimidazole.

### DETAILED DESCRIPTION OF THE PREFERRED EMBODIMENTS

The invention relates to a process for manufacture of a stable, oral, multiple-unit pharmaceutical composition containing a high concentration of benzimidazole up to about 40% w/w, and which is substantially free of, and preferably is entirely free of, micronized benzimidazole, and is substantially free, preferably entirely free, of disintegrating agents, fillers or surfactants in contact with the benzimidazole.

The term “substantially free of” a material or component means that the amount of that material or component that is present, if any, is less than the amount necessary to achieve the function otherwise attributable to the material or component if used in larger amounts. Generally, such amounts would be on the order of about 0.5% w/w to the layer or other compound. For example, “substantially free of a disintegrating agent in contact with the benzimidazole” means that the amount of compound or component used as a disintegrating agent that is in contact with the benzimidazole, if any, is an amount which is less than that which contributes to the disintegration of the composition.

The term “about,” as used herein, should generally be understood to refer to both numbers in a range of numerals. Moreover, all numerical ranges herein should be understood to include each whole integer within the range.

The term “w/w,” as used herein, should generally be understood to refer to the weight percentage of an ingredient compared to total weight of the composition, unless otherwise specifically defined.

The compositions have minimum acid degradation in 0.1N HCl after two hours and buffer release of more than about 80%-90% w/w, preferably about 85% w/w, in a pH of about 6.8 after 45 minutes. The compositions are processed in a single equipment fluid bed bottom spray processor and are capable of being filled in capsules of size 5 to size 0 for ease of administration and patient acceptance. The invention further provides a process for manufacture of a stable, oral, multiple-unit pharmaceutical composition containing a high concentration of benzimidazole up to about 35%-45% w/w, preferably up to about 40% w/w, which can be carried out continuously or in batches and is unaffected by high moisture content, which can be present in the composition. The invention also provides a process for manufacture of a stable, oral, multiple-unit pharmaceutical composition containing a high concentration of benzimidazole up to about 35%-45% w/w, preferably up to about 40% w/w, and capable of being filled in capsules of size 5 for the dose of up to 40 mg for omeprazole or up to 30 mg for lansoprazole. The invention also provides a process for manufacture of alkaline material-treated non-pariel seeds suitable for deposition of an alkaline benzimidazole dispersion containing dissolved benzimidazole, and able to withstand the rigors and attrition of a fluid bed processor. Finally, the invention also provides a process that includes spraying an aqueous or hydroalcoholic solution of benzimidazole in alkali metal hydroxide solution on treated non-pariel seeds to obtain drug pellets, which are further coated with a sealant polymer followed by an enteric polymer in a single equipment to obtain unagglomerated, uniformly-shaped and sized pellets.

More specifically, the invention relates to process for manufacture of a stable, oral, multiple-unit pharmaceutical composition containing a high concentration of benzimidazole up to about 35%-45% w/w, preferably up to about 40% w/w, without the use of micronized benzimidazole, disintegrating agents and fillers. Surfactants in these compositions are part of an enteric polymer system and are not in contact with the benzimidazole. The multiple-unit pellets of the pharmaceutical composition are in the form of unagglomerated, uniformly-shaped and sized enteric-coated pellets that are processed continuously or in batches in a single equipment fluid bed bottom spray processor. The enteric-coated pellets obtained by the process of the invention are capable of being filled in the smallest size capsules (size 5) for ease of administration and patient acceptance.

The process is environmentally friendly as it involves aqueous or hydroalcoholic media. The benzimidazole derivative is preferably selected from omeprazole, lansoprazole, rabeprazole, pantoprazole, or an optically active isomer thereof, or a mixture thereof. For the purpose of further discussing this invention herein, omeprazole is selected as the benzimidazole derivative although any of the noted benzimidazoles can be included according to the invention. The process can be carried out in batches or continuously in a single equipment to provide enteric-coated pellets. The invention involves sequential deposition of: a) an alkaline material layer on non-pariel seeds to obtain treated non-pariel seeds, b) a drug layer to obtain drug pellets, c) a sealant polymer layer to obtain sealed pellets, and d) an enteric polymer layer to obtain enteric-coated pellets. Enteric-coated pellets containing benzimidazole have minimum acid degradation after 2 hours in 0.1N HCl and a buffer release of not less than about 80%-90% w/w, preferably not less than about 85% w/w, after 45 minutes when tested in-vitro. The pellets are also capable of being filled into size 5 to size 0 capsules. This is distinct from the prior art as:
It provides a process for manufacturing pharmaceutical compositions containing a high concentration of benzimidazole up to about 35%-45% w/w, preferably up to about 40% w/w, without the use of micronized benzimidazole, disintegrating agents, fillers and surfactants in contact with the benzimidazole, having minimum acid degradation in 0.1N HCl after two hours and pH 6.8 buffer release of more than about 80%-90% w/w, preferably more than about 85% w/w, after 45 minute.
The moisture content in the formulation is less than about 5% w/w, and preferably less than about 3% w/w, without having an adverse effect on the stability of the formulation.
The process is economical and operator-friendly as it does not require micronized benzimidazole and has high batch yields with minimum drug losses as it utilizes a solution of benzimidazole for its deposition in a single equipment, thereby decreasing the chances of cross-contamination and limiting the exposure of the potent drug to the operator.

The following describe the different stages of manufacturing, which involve sequential deposition of the following layers. Other optional layers may be provided between these layers according to the invention. In a preferred embodiment, each layer is disposed immediately adjacent and over the previous layer.

Stage I: Treated Non-Pariel Seeds

The first stage in the manufacture of the pharmaceutical composition as enteric-coated pellets containing omeprazole is the deposition of an alkaline material layer on the non-pariel seeds. When an aqueous alkaline dispersion containing dissolved omeprazole was sprayed on non-pariel seeds, there was problem of breakage of the non-pariel seeds in a fluid bed bottom spray processor. It was surprisingly found that when the non-pariel seeds were treated with an alkaline material, the problem of breakage was solved. This treatment to the non-pariel seeds involves spraying an alkaline material, along with binder, on the non-pariel seeds to produce treated non-pariel seeds. Alternatively, treated non-pariel seeds can also be obtained by blending an alkaline material with starch during the production of non-pariel seeds. The alkaline material-treated non-pariel seeds that are produced possess high integrity and strength, and can withstand the further process of manufacturing enteric-coated pellets containing benzimidazole, thus leading to uniform loading of benzimidazole and resulting in high processing and batch yields. Treatment of non-pariel seeds is carried out in a fluid bed bottom processor wherein the non-pariel seeds are coated with a mixture of a water-soluble polymer, which is selected from the group including hydroxypropylmethylcellulose, and an alkaline material, for example light magnesium carbonate, and mixtures thereof. The ratio of the polymer to the alkaline material is about 1:0.1 to 0.1:1, and is preferably about 1:0.5 to 0.5:1. The total coating of this mixture is about 0.5%-6% w/w, preferably about 1%-4% w/w, of non-pariel seeds and the solid content of the spraying suspension is about 3%-20% w/w, preferably about 5%-15% w/w. The process is carried out in fluid bed processor with an inlet air temperature from about 60° C.-90° C., an outlet air temperature from about 40° C.-55° C., an atomization air pressure from about 1-3.5 bars, a fluidization flap open from about 15%-90%, and a continuous spray rate from about 1-300 ml/min. These treated non-pariel seeds are used for the production of drug pellets in a subsequent stage.

Stage II: Drug Pellets

The next stage is the deposition of a drug layer on the treated non-pariel seeds to obtain drug pellets or drug cores. The process includes the deposition of a suspension of dissolved omeprazole on the treated non-pariel seeds. The solution of omeprazole can be prepared by dissolving omeprazole in an aqueous solution of alkali metal hydroxide. The next step includes the preparation of a binder solution wherein the binder is dispersed and dissolved in water. The drug solution is mixed with the aqueous binder solution. An anti-tack agent is added to above solution. The suspension is filtered through an appropriate mesh and is sprayed on the treated non pariel seeds in a fluid bed processor with an inlet air temperature from about 60° C.-90° C., an outlet air temperature from about 40° C.-55° C., atomization air pressure from about 1-3.5 bars, fluidization flap open from about 15%-90% and continuous spray rate from about 1-300 ml/min. The drug pellets are obtained after complete spraying of an aqueous suspension containing omeprazole in dissolved form. The drug pellets are dried in fluid bed bottom spray processor to achieve a moisture content of preferably less than about 5% w/w and preferably less than about 3% w/w. The yield of the process is about 95%-99% w/w, preferably about 98% w/w.

The omeprazole content in the drug pellets is about 5%-60% w/w. The moisture content of the drug pellets is preferably less than about 5% w/w, preferably less than about 3% w/w. The particle size of the drug pellets ranges from about 600-2057 microns, and preferably from about 710-1680 microns. The particle size of the treated non-pariel seeds can be from about 420-1405 microns and preferably from about 710-1204 microns.

The alkali metal hydroxide is selected from the group that includes sodium hydroxide, potassium hydroxide, calcium hydroxide, magnesium hydroxide, ammonium hydroxide, and their mixtures thereof, and preferably includes sodium hydroxide. The concentration of sodium hydroxide in the drug pellets is from about 12%-30% w/w of omeprazole and is preferably from about 12%-25% w/w.

The binder is selected from a group of water-soluble binders which can withstand high pH, and include hydroxypropylmethylcellulose, hydroxypropylcellulose, polyvinylpyrollidone, sodiumcarboxymethylcellulose, methylcellulose, and their mixtures. Preferably, the water-soluble binder is hydroxypropylmethylcellulose alone or in combination with polyvinylpyrollidone.

Hydroxypropylmethylcellulose as used as a binder has a nominal viscosity of 5-100 cps and preferably about 5-15 cps measured on 2% w/w solution at 20° C. The concentration of the binder in the drug pellets is about 10%-40% w/w of omeprazole and is preferably about 15%-35% w/w of omeprazole.

Anti-tack agents are selected form a group that includes talc, colloidal silicon dioxide, glyceryl monostearate, glyceryl behenate, and their mixtures. Preferably the anti-tack agent includes talc with or without colloidal silicon dioxide, and is typically used in a concentration of about 5%-30% w/w, preferably from about 7.5%-25% w/w, of omeprazole.

The total solid content of the spraying suspension containing dissolved omeprazole is not more than 30% w/w and is preferably about 15-20% w/w.

The spraying suspension has a pH in the range of about 11-14.

The drug pellets, after drying, show about 100% of drug release within about 10 minutes in a pH of 6.8 buffer when tested in-vitro using a USP 24 type II dissolution apparatus.

Stage III: Sealed Pellets

The next stage is the deposition of sealant polymer layer on the drug pellets to obtain a sealed layer. A seal coating suspension is prepared by dispersing and/or dissolving a sealant polymer in water. An anti-tack agent is added to the above solution. The suspension is filtered through an appropriate mesh and is sprayed on the drug pellets in a fluid bed processor to form a seal coat, which prevents the contact of acidic enteric coating material with drug layer.

The coating parameters are same as per the drug pellet stage. The sealed pellets are dried for about 20-40 minutes to achieve the moisture level less than about 5% w/w and preferably less than about 3% w/w. The sealant polymer is selected from a group that includes hydroxypropylmethylcellulose, hydroxypropylcellulose, polyvinylpyrollidone, sodium carboxymethylcellulose, methylcellulose, and their mixtures, and is preferably hydroxypropylmethylcellulose. The concentration of hydroxypropylmethylcellulose is about 10%-150% w/w of omeprazole.

Hydroxypropylmethylcellulose as used as a sealant polymer has a nominal viscosity of about 3-100 cps and preferably about 5-15 cps measured on 2% w/w solution at 20° C.

The solid content of the above seal coating suspension is from about 6%-15% w/w, and preferably from about 8%-12% w/w. The anti-tack agent is selected from a group that includes talc, colloidal silicon dioxide, glyceryl monostearate, glyceryl behenate, and their mixtures, and used in a concentration level of about 5%-40% w/w, preferably about 10%-30% w/w, of the sealant polymer. Preferably, the anti-tack agent includes talc.

Optionally, an alkaline agent such as light magnesium carbonate can also be added to the seal coating suspension to improve the barrier property of the membrane.

The sealed pellets, after drying, shows about 100% of drug release within about 10 minutes in a pH of 6.8 buffer when tested in-vitro using a USP 24 type II dissolution apparatus.

Stage 1V Enteric-Coated Pellets

The final stage in the process for manufacture of the pharmaceutical composition as enteric-coated pellets containing omeprazole is the deposition of an enteric layer on the sealed pellets. An enteric coating suspension is prepared by dissolving a neutralizing agent in water, which is slowly added under stirring to an aqueous dispersion of methacrylic acid copolymer. This aqueous dispersion of methacrylic acid is a ready-to-use dispersion and can contain up to about 2%-5% w/w, preferably about 3% w/w, of surfactants of the total solid content present in the dispersion. Hence, the surfactants are present in the enteric polymer layer only and in no way come in contact with the omeprazole. This is in contrast to the formulations and processes in the related art as in U.S. Pat. No. 4,786,505, U.S. Pat. No. 5,385,739, U.S. Pat. No. 6,077,541, EP 1108425, and U.S. Pat. No. 6,207,198 where surfactant, in fairly large quantities (up to about 5% w/w of drug layer), is in contact with the drug. Additionally, a plasticizer, an anti-tack agent, and a colorant can be added to water to form a fine suspension, and this fine suspension is added to the above dispersion. The final enteric coating suspension is then filtered through an appropriate mesh. The pH of enteric coating suspension is about 5-6, preferably about 5.2-5.8. The sealed pellets, after drying, are coated with the enteric coating suspension. The coating parameters are same as drug pellets stage. The enteric coat polymer is methacrylic acid copolymer and is used in a concentration of about 10%-35% w/w, preferably about 12.5-30% w/w, of the sealed pellets.

The plasticizers are selected from a group that includes polyethylene glycol, triethyl citrate, triacetin, tributyl citrate, castor oil, dibutyl sebacate, and polysorbate, e.g., TWEEN 80, and mixtures thereof, and is used in a concentration of about 5%-30% w/w, preferably about 10%-25% w/w, of the enteric coat polymer. A preferred plasticizer includes polyethylene glycol. The neutralizing agent is selected from a group that includes sodium hydroxide, potassium hydroxide, calcium hydroxide, magnesium hydroxide, ammonium hydroxide, and their mixtures, and preferably includes sodium hydroxide and is used in a concentration of about 0.5% to 4% w/w, more preferably about 1%-2% w/w of the enteric coat polymer. Any other agent that is capable of neutralizing the acidic group of methacrylic acid copolymer can also be used.

The anti-tack agent is selected from a group that includes talc, colloidal silicon dioxide, glyceryl monostearate, glyceryl behenate, and their mixtures, and can be used in a concentration level of about 5%-35% w/w, preferably about 10%-30% w/w, of the polymer for enteric coating. The preferable choice is talc, with or without colloidal silicon dioxide. The solid content of the final enteric coating suspension is in the range of about 10%-35% w/w, preferably about 15%-25% w/w.

The amount of enteric polymer guarantees gastric resistance and allows the dissolution of omeprazole in the proximal part of the small intestine. The final enteric-coated product is sieved through an appropriate mesh and is capable of being encapsulated in hard gelatin capsules, preferably from size 5 to size 0. The entire manufacturing process is aqueous and it results in spherical, glossy pellets containing a negligible amount of twins or triplets. The benzimidazole content in the final enteric-coated pellets is about up to about 35%-45% w/w, and preferably up to about 40% w/w. The yield of the final product is about in the range of about 95%-99.9% w/w and usually above about 98% w/w.

In one of the embodiments of the invention, the process involving all the stages, i.e., the treated non-pariel seed stage, the drug pellet stage, the sealed pellet stage, and the enteric-coated pellet stage, is carried out continuously in a single equipment fluid bed processor.

In another embodiment of the invention, the process is a batch process, but all stages, i.e., the treated non-pariel seed stage, the drug pellet stage, the sealed pellet stage, and the enteric-coated pellet stage, are preferably carried out in a single fluid bed processor, where representative samples are sampled at the end of each stage.

Other equipment, such as a coating pan or a tangential spray coater can also be used for manufacturing enteric-coated pellets containing benzimidazole using the above process.

### EXAMPLES

The invention is further described by the following non-limiting Examples, which are merely representative of certain embodiments of the claimed invention. Enteric-coated pellets containing omeprazole involve the following manufacturing stages, which are as follows. The examples were prepared as described above generally, with specifics described below.

Stage I: Preparation of Treated Non-Pariel Seeds

| Sr. No. | Ingredients | Quantity (kg) |
|---|---|---|
| 1 | Non-pariel seeds 18-20# | 100 |
| 2 | Hydroxypropylmethylcellulose | 1.5 |
| 3 | Magnesium Carbonate | 1.5 |
| 4 | Water | 30 |

Hydroxypropylmethylcellulose (HPMC) was dispersed and dissolved in water. Magnesium carbonate was added to this solution to obtain a fine suspension, which was filtered through an appropriate mesh. The fine suspension was sprayed on non-pariel seeds in a fluid bed bottom spray processor to obtain treated non-pariel seeds, which were used for further processing.

Hydroxypropylmethylcellulose and magnesium carbonate were used in the ratio of about 1:0.5 to 0.5:1 and were used in a concentration level of about 1-4% w/w of the non-pariel seeds.

### Example 1

### Omeprazole Enteric Coated Pellets

| | | | | | |
|---|---|---|---|---|---|
| Sr. | | Quantity | Quantity | Quantity | Quantity |
| No. | Ingredients | (% w/w) | (% w/w) | (% w/w) | (% w/w) |

Stage II: Drug Pellets

| | | | | | |
|---|---|---|---|---|---|
| 1. | Omeprazole | 24.5 | 13.8 | 8.5 | 40.00 |
| 2. | Treated Non- | 32.3 | 56.5 | 61.9 | 15.00 |
| | Pariel Seeds | | | | |
| 3. | Sodium | 3.2 | 1.8 | 1.1 | 5.26 |
| | Hydroxide | | | | |
| 4. | Talc | 3.7 | 2.1 | 1.3 | 3.00 |
| 5. | HPMC E15 | 7.3 | 3.4 | 2.6 | 6.00 |
| 6. | Water | Qs | Qs | Qs | Qs |

Stage III: Sealed Pellets

| | | | | | |
|---|---|---|---|---|---|
| 7. | HPMC E5 | 4.3 | 4.7 | 4.9 | 4.35 |
| 8. | Talc | 0.4 | 0.5 | 0.5 | 0.47 |
| 9. | Water | Qs | Qs | Qs | Qs |

Stage IV: Enteric Coated Pellets

| | | | | | |
|---|---|---|---|---|---|
| 10. | Methacrylic acid | 17.3 | 12.4 | 13.7 | 18.5 |
| | copolymer type C | | | | |
| | (solid content) | | | | |
| 11. | Polyethylene | 1.7 | 1.2 | 1.4 | 1.9 |
| | Glycol 6000 | | | | |
| 12. | Talc | 3.8 | 2.6 | 2.9 | 3.9 |
| 13. | Titanium dioxide | 1.2 | 0.8 | 1.0 | 1.3 |
| 14. | Sodium | 0.3 | 0.2 | 0.2 | 0.32 |
| | hydroxide | | | | |
| 15. | Water | Qs | Qs | Qs | Qs |
| | particle size of | 18-20 | 16-18 | 16-18 | 20-22 |
| | treated non-pariel | mesh | mesh | mesh | mesh |
| | seeds | ASTM | ASTM | ASTM | ASTM |
| | Dose | 20 mg | 20 mg | 20 mg | 40 mg |
| | Size of capsule | 5 | 4 | 2 | 5 |
| | for | | | | |
| | encapsulation | | | | |

Stage II: Drug Pellets

Sodium hydroxide was dissolved in water and then cooled to room temperature. Omeprazole was then added to this solution. Separately, hydroxypropylmethylcellulose E15 was dissolved or dispersed in water. The omeprazole solution was mixed with the hydroxypropylmethylcellulose solution. Next, talc was added to the solution and the resulting suspension was filtered through an appropriate mesh. The suspension was sprayed on the treated non-pariel seeds in a fluid bed bottom spray processor to obtain the drug pellets, which were dried in the same equipment to a moisture content of less than 5% w/w and preferably less than 3% w/w. After drying, the drug pellets have about 100% of drug release within about 10 minutes in a pH of 6.8 buffer when tested in-vitro using a USP 24 type II dissolution apparatus.

Stage III: Sealed Pellets

A seal coating suspension was prepared by dispersing and/or dissolving hydroxypropylmethylcellulose E5 in water. Talc was then added to the solution. The resulting suspension was filtered through an appropriate mesh and sprayed on the drug pellets in a fluid bed processor to form sealed pellets. These sealed pellets were dried to a moisture content of less than 5% w/w and preferably less than 3% w/w. After drying, the sealed pellets have about 100% of drug release within about 10 minutes in a pH of 6.8 buffer when tested in-vitro using a USP 24 type II dissolution apparatus.

Stage IV: Enteric-Coated Pellets

An enteric coating suspension was prepared by dissolving sodium hydroxide in water, which was slowly added under stirring to an aqueous dispersion of methacrylic acid copolymer type C. Polyethylene glycol 6000 was separately dissolved in water. Talc and titanium dioxide were added to the solution to form a fine suspension and this suspension was added to the above dispersion. The final enteric coating suspension was then filtered through an appropriate mesh. This suspension was sprayed on the sealed pellets in a fluid bed processor to obtain enteric-coated pellets containing omeprazole. These enteric-coated pellets were then dried to a moisture content of less than 5% w/w and preferably less than 3% w/w.

The process of the invention results in the production of enteric-coated pellets containing omeprazole that does not require micronization of omeprazole or any additional excipients like disintegrating agents, fillers or surfactants in contact with omeprazole for improving the dissolution of omeprazole.

The enteric-coated pellets containing omeprazole thus obtained were tested in vitro for acid resistance in 0.1 N HCl after 2 hours and buffer dissolution in a pH of 6.8 after 45 minutes which is described below. The acceptance criteria for these pellets are described below.

The pellets can receive an additional coat of an enteric layer if it does not comply with the acid degradation criteria as specified below.

The enteric-coated pellets containing omeprazole can fill capsules of varying sizes from size 5 to size 0 depending on the final concentration of omeprazole in the enteric-coated pellets.

### Example 2

### Lansoprazole Enteric-Coated Pellets

The formulation for encapsulation in size 5 hard gelatin capsules is as follows.

| | | |
|---|---|---|
| | | Quantity |
| Sr. No. | Ingredients | (% w/w) |

Stage I: Drug Pellets

| | | |
|---|---|---|
| 1. | Lansoprazole | 30.0 |
| 2. | Treated Non-Pariel Seeds 18-20 mesh ASTM | 22.7 |
| 3. | Sodium Hydroxide | 7.5 |
| 4. | Talc | 3.0 |
| 5. | Hydroxypropylmethylcellulose E15 | 6.0 |
| 6. | Colloidal Silicon dioxide | 0.5 |
| 7. | Water | QS |
| 8. | Isopropyl Alcohol | QS |

Stage II: Sealed
Pellets

| | | |
|---|---|---|
| 9. | Hydroxypropylmethylcellulose E5 | 4.2 |
| 10. | Talc | 0.4 |
| 11. | Water | Qs |

Stage III: Enteric
Coated Pellets

| | | |
|---|---|---|
| 12. | Methacrylic acid copolymer type C (solid | 18.6 |
| | content) | |
| 13. | Polyethylene Glycol 6000 | 1.86 |
| 14. | Talc | 3.9 |
| 15. | Colloidal Silicon dioxide | 0.24 |
| 16. | Titanium dioxide | 0.85 |
| 17. | Sodium hydroxide | 0.25 |
| 18. | Water | Qs |
| | Size of capsule for encapsulation | ‘5’ |
| | Dose | 30 mg |

The process for the manufacture of enteric-coated pellets containing lansoprazole was performed as described above except that lansoprazole was dissolved in a sodium hydroxide solution in water containing a small amount of isopropyl alcohol.

In-vitro dissolution studies of these pellets were carried out as described below. The acceptance criteria for these pellets are also described below. The pellets can receive an additional coat of an enteric layer if they would otherwise not comply with specified acid degradation criteria.

Dissolution Studies:

The pellets containing omeprazole or lansoprazole, which were manufactured as described above were tested for acid resistance in 0.1 N HCl for 2 hours and a buffer dissolution in a pH of 6.8 for 45 minutes using a USP 24 Type II dissolution test apparatus. The results obtained were as follows.

| Time | pH | % Drug release | Specification |
|---|---|---|---|
| 2 hours | 0.1 N HCI | 0-7 | NMT 15% degrades in 2 |
| | | | hours |
| 45 minutes | pH 6.8 buffer | 90-100 | NLT 85% |

The pellets that meet the requirements of the specification were filled in capsules of different sizes containing a unit dose of benzimidazole.

The pellets containing up to about 40% w/w of omeprazole were able to fill size 5 capsules for a dosage of up to about 40 mg of omeprazole. The enteric-coated pellets containing about 30% w/w of lansoprazole were able to fill size 5 capsules for a dosage of up to about 30 mg of lansoprazole.

Stability:

The enteric-coated pellets containing about 20 mg omeprazole were prepared as described above and encapsulated in size 5 hard gelatin capsules and subjected to an accelerated stability condition at 40° C./75% RH and 25° C./60% RH. The stability results were as follows.

| | | | Dissolution Profile | |
|---|---|---|---|---|
| | | Assay | Acid Stage | Buffer Stage |
| Period | Condition | (% w/w) | (% w/w) | (% w/w) |
| Initial | | 98.5 | 1.0 | 99.85 |
| 1 Month | 25° C./60% RH | 99.9 | 0.24 | 98.68 |
| 1 Month | 40° C./75% RH | 100.05 | 0.0 | 99.04 |
| 3 Month | 25° C./60% RH | 97.25 | 1.5 | 98.45 |
| 3 Month | 40° C./75% RH | 99.7 | 0.31 | 97.56 |
| 6 Month | 25° C./60% RH | 98.45 | 0.1 | 101.1 |
| 6 Month | 40° C./75% RH | 96.15 | 0.0 | 92.59 |

The results show that enteric-coated pellets containing omeprazole of the present invention are stable for at least 2 years.

Bioequivalence Study:

A bioequivalence study was carried out in 12 healthy human volunteers using Losec RTM as a reference. The results were as follows.

| Pharmacokinetic parameters | Test | Reference |
|---|---|---|
| Cmax (μg/ml) (Avg. ± std. dev.) | 0.92(±0.22) | 0.88(±0.17) |
| Tmax (hrs.) (Avg. ± std. dev.) | 3.67(±0.47) | 3.25(±0.43) |
| AUC (0-24) (μg · hr/ml) (Avg. ± std. dev.) | 6.70(±1.39) | 7.70(±1.62) |

Various modifications and alterations can be made in the process and the product without varying the spirit and scope of the invention. The process and the product described in the invention are merely illustrative of the preferred embodiments of the invention and do not limit the scope of the invention.

1. A process for manufacture of a pharmaceutical composition, which comprises:
depositing, on non-pariel seeds, an alkaline material layer comprising a water insoluble alkaline material to obtain treated non-pariel seeds with increased resistance to breakage;
depositing, on the treated non-pariel seeds, a drug layer comprising benzimidazole in an amount of up to about 40% w/w of the composition and being substantially free of propylene glycol, to obtain drug pellets;
depositing, on the drug pellets, a sealant polymer layer which is substantially free of propylene glycol, to obtain sealed pellets; and
depositing, on the sealed pellets, an enteric polymer layer containing surfactants to obtain a pharmaceutical composition comprising enteric coated pellets which are substantially free of surfactants, disintegrating agents, or fillers in contact with the benzimidazole.

2. The process of claim 1, wherein the treated non-pariel seeds are prepared by depositing on the non-pariel seeds, a mixture of hydroxypropylmethylcellulose and magnesium carbonate in a ratio of about 1:0.1 to about 0.1:1 and in an amount of about 1% to 4% by weight of non-pariel seeds.

3. The process of claim 1, wherein the drug layer is deposited by:
dissolving an alkali metal hydroxide in aqueous or hydroalcoholic media to form an alkaline solution;
dissolving benzimidazole in the alkaline solution;
dispersing and/or dissolving a binder in an aqueous media;
mixing the alkaline benzimidazole solution with the binder solution to form a benzimidazole-binder dispersion;
adding an anti-tack agent to the benzimidazole-binder dispersion to form a benzimidazole suspension; and
spraying the benzimidazole suspension in dissolved form on the treated non-pariel seeds followed by drying to obtain the drug pellets.

4. The process of claim 3, wherein the drug layer comprises the alkali metal hydroxide in an amount about 12% to 30% w/w of the benzimidazole, the binder in an amount of about 10% to 40% w/w of the benzimidazole, the anti-tack agent in an amount of about 7.5% to 25% w/w of the benzimidazole, wherein the alkali metal hydroxide is selected from the group consisting of sodium hydroxide, potassium hydroxide, calcium hydroxide, magnesium hydroxide, ammonium hydroxide, and mixtures thereof, the binder is selected from the group consisting of hydroxypropylmethylcellulose, hydroxypropylcellulose, methylcellulose, sodiumcarboxymethylcellulose, polyvinylpyrollidone, and mixtures thereof, and the anti-tack agent is selected from the group consisting of talc, colloidal silicon dioxide, glyceryl monostearate, glyceryl behenate, and mixtures thereof.

5. The process of claim 1, wherein the sealant polymer layer is deposited by:
forming a suspension of sealant polymer(s) and an anti-tack agent in an aqueous media; and
seal coating the drug pellets by spraying the suspension thereon, followed by drying to obtain the sealed pellets.

6. The process of claim 5, wherein the sealant polymer is present in an amount of about 10% to 150% w/w of the benzimidazole, and comprises one or more of hydroxypropylmethylcellulose, hydroxypropylcellulose, methylcellulose, sodium carboxymethylcellulose, polyvinylpyrollidone, and the anti-tack agent is present in an amount of about 10% to 30% w/w of the sealant polymer and comprises talc, colloidal silicon dioxide, glyceryl monostearate, glyceryl behenate and mixtures thereof.

7. The process of claim 1, wherein the enteric polymer layer is deposited by:
dissolving a neutralizing agent in aqueous media to form a solution;
adding the neutralizing agent solution to an aqueous dispersion of the enteric polymer to form a dispersion;
forming a suspension of a plasticizer, anti-tack agent and, optionally, a coloring agent, in aqueous media;
mixing the suspension and dispersion to form an enteric coating suspension having a pH of about 5.2 to 5.8; and
spraying the enteric coating suspension onto the sealed pellets, followed by drying to obtain the enteric coated pellets.

8. The process of claim 7, wherein the enteric polymer is methacrylic acid copolymer and is present in an amount of about 12.5% to 30% w/w of the sealed pellets, the neutralizing agent is present in an amount of about 1% to 2% w/w of the enteric polymer, the plasticizer is present in an amount of about 10% to 25% w/w of the enteric polymer, the anti-tack agent is present in an amount of about 10% to 30% w/w of the enteric polymer, and wherein the neutralizing agent is selected from the group consisting of sodium hydroxide, potassium hydroxide, calcium hydroxide, magnesium hydroxide and ammonium hydroxide, and mixtures thereof, the plasticizer is selected from the group consisting of polyethylene glycol, triethyl citrate, triacetin, tributyl citrate, castor oil, dibutyl sebacate, and polysorbate, and mixtures thereof, and the anti-tack agent is selected from the group consisting of talc, colloidal silicone dioxide, glyceryl monostearate, glyceryl behenate, and mixtures thereof.

9. The process of claim 1, wherein the benzimidazole is selected from the group consisting of omeprazole, lansoprazole, and mixtures thereof, and the pharmaceutical composition is in the form of stable oral multiple unit pellets capable of being filled into size 5 to size 0 capsules.

10. The process of claim 9, wherein the stable oral multiple unit pellets have a moisture content of not more than 5% w/w, the benzimidazole is non-micronized and is omeprazole or lansoprazole, and the multiple unit dose pellets contain up to about 40 mg omeprazole or up to about 30 mg lansoprazole, and are of a size to fill a size 5 capsule.

11. The process of claim 1, wherein the process is carried out in a fluid bed processor at an inlet air temperature about 60° C. to 90° C., an outlet air temperature about 40° C. to 55° C., an atomization air pressure about 1 to 3.5 bars, a fluidization flap open to about 15% to 90%, and a spray rate about 1 to 300 ml per minute.

12. The process of claim 1, wherein the process is a batch or continuous process and is carried out in a single equipment fluid bed bottom spray processor.

13. A pharmaceutical composition obtainable from the process of claim 1.

14. A pharmaceutical composition obtained from the process of claim 1.

15. A stable oral multiple unit pharmaceutical composition in the form of enteric coated pellets comprising non-pariel seeds coated with an alkaline material layer comprising a water insoluble alkaline material; a drug layer, disposed over the alkaline material layer, comprising benzimidazole in an amount of up to about 40% w/w of the composition and being substantially free of propylene glycol; a sealant polymer layer, disposed over the drug layer, which is substantially free of propylene glycol; and an enteric polymer layer, disposed over the sealant polymer layer, containing surfactants; wherein the pharmaceutical composition is substantially free of surfactants, disintegrating agents, or fillers in contact with the benzimidazole.

16. The pharmaceutical composition of claim 15, wherein the alkaline material layer comprises a mixture of hydroxypropylmethylcellulose and magnesium carbonate in the ratio of about 1:0.1 to 0.1:1 and is present in an amount of about 1 to 4% by weight of non-pariel seeds.

17. The pharmaceutical composition of claim 15, wherein the drug layer comprises an alkali metal hydroxide in an amount about 12% to 30% w/w of the benzimidazole, a binder in an amount of about 10% to 40% w/w of the benzimidazole, and an anti-tack agent in an amount of about 7.5% to 25% w/w of the benzimidazole.

18. The pharmaceutical composition of claim 17, wherein the alkali metal hydroxide is selected from the group consisting of sodium hydroxide, potassium hydroxide, calcium hydroxide, magnesium hydroxide, ammonium hydroxide, and mixtures thereof, and is present in an amount of about 12% to 25% w/w of benzimidazole, the binder is selected from the group consisting of hydroxypropylmethylcellulose, hydroxypropylcellulose, methylcellulose, sodiumcarboxymethylcellulose, polyvinylpyrollidone, and mixtures thereof, and is present in an amount of about 15%-35% w/w of the benzimidazole, and the anti-tack agent is selected from the group consisting of talc, colloidal silicon dioxide, glyceryl monostearate, glyceryl behenate, and mixtures thereof, and is present in an amount of about 7.5% to 25% w/w of the benzimidazole.

19. The pharmaceutical composition of claim 15, wherein the sealant polymer layer is present in an amount of about 10% to 150% w/w of the benzimidazole, and comprises an anti-tack agent present in an amount of about 10% to 30% w/w of sealant polymer.

20. The pharmaceutical composition of claim 19, wherein the sealant polymer is selected from the group consisting of hydroxypropylmethylcellulose, hydroxypropylcellulose, methylcellulose, sodium carboxymethylcellulose, polyvinylpyrollidone, and mixtures thereof, and the anti-tack agent is selected from the group consisting of talc, colloidal silicon dioxide, glyceryl monostearate, glyceryl behenate, and mixtures thereof.

21. The pharmaceutical composition of claim 15, wherein the enteric layer is present in an amount of about 12.5% to 30% w/w of the composition prior without the enteric coating, and comprises a neutralizing agent present in an amount of about 1% to 2% w/w of enteric polymer, a plasticizer present in an amount of about 10% to 25% w/w of enteric polymer, and an anti-tack agent present in an amount of about 10% to 30% w/w of enteric polymer

22. The pharmaceutical composition of claim 21, wherein the enteric polymer is methacrylic acid copolymer, the neutralizing agent is selected from the group consisting of sodium hydroxide, potassium hydroxide, calcium hydroxide, magnesium hydroxide, ammonium hydroxide, and mixtures thereof, the plasticizer is selected from the group consisting of polyethylene glycol, triethyl citrate, triacetin, tributyl citrate, castor oil, dibutyl sebacate, and polysorbate, and the anti-tack agent is selected from the group consisting of talc, colloidal silicone dioxide, glyceryl monostearate, glyceryl behenate, and mixtures thereof.

23. The pharmaceutical composition of claim 15, wherein the benzimidazole is selected from the group consisting of omeprazole, lansoprazole, and their mixtures thereof and the composition has a minimum acid degradation and buffer release of not less than 85% after 45 minutes and is sized to fill a size 5 to size 0 capsule.

24. The pharmaceutical composition of claim 17, having a moisture content of not more than 5% w/w, wherein the benzimidazole is omeprazole or lansoprazole, and the composition contains up to about 40 mg omeprazole or up to about 30 mg lansoprazole, and is of a size to fill a size 5 capsule.

25. The pharmaceutical composition of claim 15, having a moisture content of not more than 5% w/w and wherein the benzimidazole is non-micronized.
